(19) United States
(12) Patent Application Publication
(10) Pub. No.: US 2004/0071762 A1
(43) Pub. Date: Apr. 15, 2004

(54) Diagnostic test for schizophrenia, using niacin

(75) Inventors: Alexander Iain Munro Glen, Kincraig (GB); Pauline Elizabeth Ward, Inverness (GB); David Frederick Horrobin, Stirling (GB)

Correspondence Address: JACOBSON HOLMAN PLLC, 400 SEVENTH STREET N.W., SUITE 600, WASHINGTON, DC, 20004, US

(73) Assignee: Scotia Holdings PLC

(21) Appl. No.: 10/384,065

(22) Filed: Mar. 10, 2003

Related U.S. Application Data
(63) Continuation of application No. 09/147,113, filed on Apr. 6, 1999.
Application No. 09/147,113 is a 371 of international application No. PCT/GB97/01461, filed on May 29, 1997.

(30) Foreign Application Priority Data
May 29, 1996 (GB) 9610971.5

(51) Int. Cl. A61K 31/455; A61K 9/70

(52) U.S. Cl. 424/449; 514/356

(57) ABSTRACT

Niacin in the form of a topical preparation, both as such and when incorporated in devices for application to the skin; the devices themselves; diagnosis or monitoring of schizophrenia using the preparations or devices; and manufacture of medicaments for such monitoring and diagnosis.

### FIELD OF INVENTION

The invention relates to a diagnostic test for use in schizophrenia.

### BACKGROUND

Schizophrenia is a common psychiatric disease which in most populations affects about 1% of the population. There are many different clinical presentations of the disease which can be broadly divided into two main groups, the “positive” features and the “negative” or “deficit” features. The positive features include auditory hallucinations, bizarre behaviour, thought disorder and sometimes paranoia. The negative features include social and emotional withdrawal and an absence of emotional responses to what is happening in the individual's environment. Some of these features are shared by other psychiatric disorders and, especially in the early years of the disease, it may be difficult to distinguish schizophrenia from depression, manic-depression, drug-induced psychoses and psychoses related to alcoholism. Only the long term course allows a diagnosis of schizophrenia to be made with reasonable certainty. This diagnostic uncertainty in the early stages of the disease has important practical consequences because therapies for the different disorders differ and the uncertainty may delay, sometimes for many years, the introduction of optimum therapy. A biochemically based test which would improve early diagnosis would therefore be of great value.

There is evidence that in schizophrenia there is a biochemical abnormality in the metabolism of the fatty acids of membrane phospholipids to prostaglandins (Horrobin D F, Lancet 1:936-7, 1977; Horrobin D F, Glen A I M, Vaddadi K S, Schizophrenia Research, 1994). In particular, in schizophrenics with the deficit syndrome there is a substantial reduction in the concentrations of arachidonic acid (AA) and docosahexaenoic acid (DHA) in red cell membranes (European Patent Application 0599576). There are also peculiarities in the cPLA2 (phospholipase A2) gene structure (PCT patent application No.97/04127).

Niacin (nicotinic acid) is a vitamin which, when given in a large oral dose, causes marked flushing of the skin over the head, upper body and arms. This flushing is due to vasodilation caused by the release of prostaglandin D₂ from AA. In 1980, Horrobin noted that whereas most normal individuals flush markedly in response to 100-300 mg niacin given orally, a substantial proportion of schizophrenics fail to flush. Many but not all of the non-flushing individuals were patients with the deficit syndrome. Horrobin therefore suggest that a flushing response to oral niacin might be used as to assist diagnosis of schizophrenia (Horrobin D F, Journal of Orthomolecular Psychiatry, 9: 33-34, 1980). The idea was that individuals who showed a pattern of behaviour possibly indicative of schizophrenia and who failed to flush in response to niacin would almost certainly have schizophrenia. Individuals who did flush might or might not have the disease. However, in non-flushers appropriate therapy could be introduced without delay. Horrobin also noted that some patients whose schizophrenia improved on being treated with essential fatty acids shifted from a non-flushing to a flushing response and he suggested that flushing could be used as an indicator of improvement in response to treatment. Other investigators have confirmed that a substantial proportion of schizophrenics fail to respond to a fixed oral dose of niacin by flushing (Rybakowski J et al, Biological Psychiatry, 29: 834-61, 1990, Hudson J et al, Biological Psychiatry, 41: 507-13, 1997). However, there are several drawbacks to the oral test. The flushing reaction is usually accompanied by pricking and tingling sensations which can be quite distressing and there can be a fall in blood pressure. In particular, in paranoid patients the responses may arouse suspicion and hostility. Further, the oral treatment involves a single, all or nothing response which is difficult if not impossible to quantify.

### THE INVENTION

We have now surprisingly found that the oral test can be replaced by a simple test involving the application of different concentrations of niacin in any effective form, or even a single chosen concentration, to the skin. This is easy to apply, and causes no distress. The test is quickly read, for example in 3 to 6 minutes with a negative reaction desirably checked after 15 minutes in contrast to the 30-60 minutes which may be required for the oral test. Further, it is not affected by whether or not the individual being tested has a full stomach, and it can be made quantitative.

The invention in its various aspects is set out fully in the claims, to which reference should be made, but may be summarised as niacin in the form of topical preparations, both as such and when incorporated in devices for application to the skin; the devices themselves; diagnosis or monitoring of schizophrenia using the preparations or devices; and manufacture of medicaments for such monitoring and diagnosis.

The test involves the topical application of niacin solutions, preferably in a carrier such as gauze, cotton, wax or appropriate fibre or absorbent material, to the skin of any part of the body, but conveniently the forearm or upper arm. A range of solutions may be used from 0.00001M to 1 molar. A single concentration may be chosen and used but desirably use is made of a range of steps varying from two to twenty. We have found it convenient is to use a range of four solutions of 0.1, 0.01, 0.001 and 0.0001M, e.g. 0.05 ml of each, but other concentrations within the range and other numbers of steps can be used as found appropriate, both in diagnosis and in monitoring as discussed above. It is particularly convenient to incorporate the niacin-impregnated patches into a plastic or other strip, wherein the individual patches range for example from 1 to 20 mm in diameter, and wherein the patches are in individual wells or depressions which may be anything from 0.5 mm to 20 mm apart, preferably 2-10 mm apart. The wells or depressions may be sealed by another plastic or other strip which isolates them from the atmosphere and which can be removed immediately prior to testing. The strip with the open wells can then be applied to the skin surface with light pressure and removed after an appropriate time which may be e.g. 0.5 minutes up to 10 or 15 minutes but preferably 3 to 6 minutes.

In normal individuals, after a short time, an adequate topical concentration of niacin produces both redness (flushing) of the skin and skin swelling (oedema). Both of these effects are due to vasodilatation of the small blood vessels of the skin. The redness may be scored in relation to the adjacent unaffected skin on an appropriate scale which can be either “present” or “absent”, or can be semi-quantitative, such as mild, moderate or marked, or can be made fully quantitative by use of a spectrophotometer or other appropriate instrument e.g. a blood flow meter. The oedema can likewise be scored as present or absent or can be scored semi-quantitatively or quantitatively.

We have applied this test to 38 patients with schizophrenia and 22 controls, using niacin concentrations of 0.1, 0.01, 0.001 and 0.0001M, 0.05 nil of each, and time intervals of 0, 5, 10, 15, 20 and 25 minutes after application of the niacin containing patches. We have also used a scoring system in which the degree of redness under each patch was scored as 0=no change, 1=mild redness, 2=moderate redness and 3=marked redness. When the test is used commercially, sample colour charts may show how the test can be used with individuals of varying skin colour. With particularly dark-skinned individuals, oedema or a measurement of skin surface temperature or heat loss may be required to give the best results.

Table 1 shows the scores obtained in the two groups at 5 minutes after application of the test, a time showing good discrimination between the groups:

Table 1 Percentages of normal controls (n=22) and of schizophrenics (n=38) responding at 5 minutes to the application of topical niacin with absent [0], mild [1], moderate [2] or marked [3] reddening of the skin, C=controls and S=schizophrenics.

Niacin Concentrations

| | 0.1 M | | 0.01 M | | 0.001 M | | 0.000. M | |
|---|---|---|---|---|---|---|---|---|
| Response | C | S | C | S | C | S | C | S |
| Absent [0] | 4.5 | 13.5 | 9.1 | 41.2 | 31.8 | 67.6 | 35.3 | 88.2 |
| Mild [1] | 9.1 | 37.8 | 13.6 | 41.2 | 9.1 | 29.4 | 29.4 | 8.8 |
| Moderate [2] | 27.3 | 43.2 | 45.5 | 17.6 | 40.9 | 2.9 | 17.6 | 2.9 |
| Marked [3] | 59.1 | 5.4 | 31.8 | 0 | 18.2 | 0 | 17.6 | 0 |

As may be seen, the three lower concentrations do not give a marked reaction in any of the schizophrenics. Even a moderate reaction, at the two lower concentrations, is shown by very few of the schizophrenics, the great majority showing a mild reaction or none. In contrast, only 4.5% of the normal individuals failed to give a response even to the highest concentration. Given the difficulty of making a secure diagnosis of schizophrenia, the indication given by the new test is thus highly valuable, for use in conjunction with other criteria. A valuable feature is that the test, as with oral niacin (Rybakowski J et al, Biological Psychiatry, loc. cit.) may be expected to give a response in depressives the same as for normal individuals.

Suitably the niacin is in aqueous solution in the form of a C₁ to C₁₀ alkyl ester such as the methyl or hexyl ester but any effective form that is to say a form passing the skin and eliciting the flushing reaction in normal individuals, may be used. Nicotinamide (niacinamide), which does not elicit the reaction, is not suitable.

### IN THE DRAWINGS

FIG. 1 is a simple device for application by hand;

FIG. 2 is a cuff-style device with a hook and loop (“Velcro” trade mark) fastening; and

FIG. 3 is a section of part of FIG. 1, taken axially of the strip at the right hand end as seen.

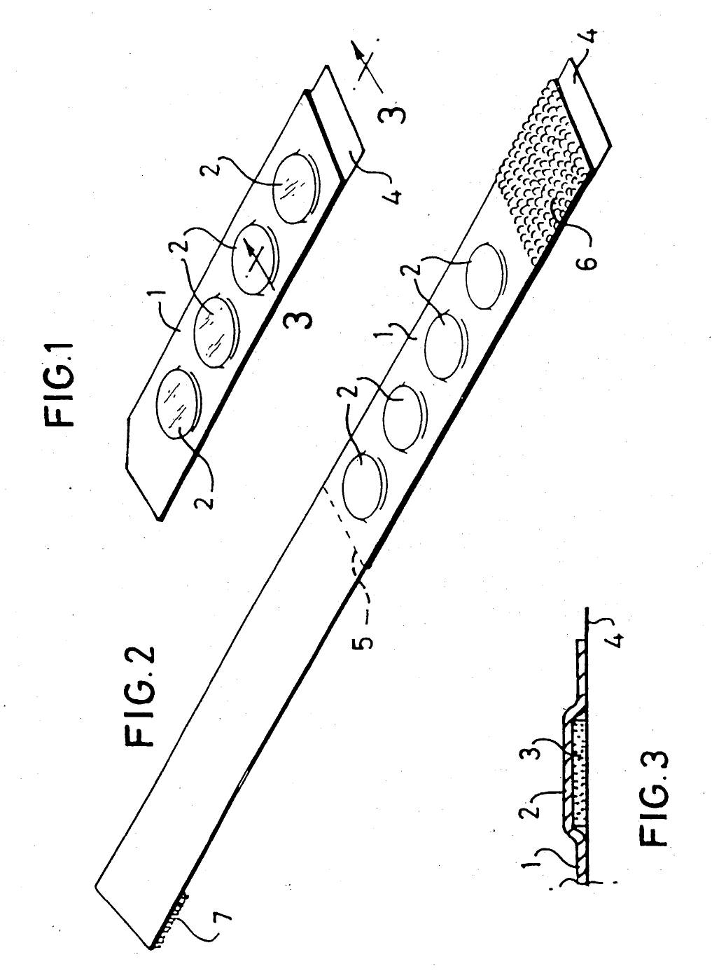

In the drawings, which are merely of examples of ways of using the invention, a moulded flexible plastics strip 1 has wells 2, each holding a gauze pad 3 impregnated with methyl nicotinate solution at the successive concentrations of Table 1. The pads are 16 mm diameter and approximately 1.5 mm thick. A backing strip 4 is secured by pressure sensitive adhesive, to be peeled off when the device is to be used. After peeling, the device is applied to the body, conveniently to the forearm, with the pads in contact with the skin. The device may be held under finger pressure (FIG. 1, one finger to a pad) or by the fastening (FIG. 2), the two parts of which are indicated at 6 and 7, and the extent of the backing strip at 5. After for example 5 minutes, the device is removed and the skin reaction assessed as already described.

1. Niacin as a topical preparation giving absorption of the niacin in any effective form through the skin, with production of a differential effect, as between schizophrenics and non-schizophrenics, in redness and swelling of the skin.

2. A preparation as in claim 1, wherein the niacin is in the form of a C₁₋₁₀ alkyl ester particularly C₁-C₆ and most particularly methyl.

3 A preparation as claim 1 or 2, presented as two or more sub-preparations with the niacin in differing concentrations.

4. A preparation as in claim 1, 2 or 3, held in a carrier in a sealed device which, after removal of the seal, can be applied to the skin to bring the preparation into contact with the skin.

5. A method of manufacture of a medicament for the diagnosis or monitoring of schizophrenia by observation of a differential effect in redness and swelling of the skin as between schizophrenics and non-schizophrenics, wherein niacin in any effective form is incorporated in a topical preparation giving absorption of the niacin through the skin.

6. Diagnosis or monitoring of schizophrenia by observation of a differential effect in redness and swelling of the skin as between schizophrenics and non-schizophrenics, following application of niacin in any effective form in a topical preparation giving absorption of the niacin through the skin.

7. A method as in claim 5, or diagnosis or monitoring as in claim 6, wherein the preparation is as set out in claim 3 or 4.

8. Method, or diagnosis or monitoring, as in claims 5 to 7, wherein the redness is observed visually in terms of colour or by temperature measurement or heat loss, and is scored against a pre-prepared scale.

9. Preparation, method, or diagnosis or monitoring as claimed above, using concentrations(s) of niacin 0.0001 to 0.1 M.

10. Preparation method, or diagnosis or monitoring as claimed above, with observation of the effect after 0.5 to 10 minutes following application to the skin, more conveniently 3 to 6 minutes, with a negative response optionally checked after 15 minutes.

11. Preparation, method or diagnosis or monitoring as claimed above, using a preparation held as in claim 4, wherein the device is a flexible strip holding the preparation in a well, or a plurality of spaced wells with differing niacin concentrations, and the seal is a removable cover film the flexible strip being suited after removal of the film for application to the skin with the carrier in contact with the skin.

12. As such, the device set out in claim 4 or 11, optionally in association with a colour chart, for scoring skin flushing reaction, or other pre-prepared quantifying aid.
